(19) United States
(12) Patent Application Publication
(10) Pub. No.: US 2019/0022590 A1
(43) Pub. Date: Jan. 24, 2019

(54) DEVICE FOR SEPARATION AND PURIFICATION OF COLLAGEN TYPE 2 IN CHICKEN BONES

(71) Applicants: Titan Biological & Agricultural Technology Co., Ltd., Apia (WS); Super Well Biotechnology Corporation, Taichung (TW)

(72) Inventors: Zer-Ran Yu, Taichung (TW); Hui-Chen Kuo, Taichung (TW); Be-Jen Wang, Taichung (TW); Po-Wen Yu, Taichung (TW); Hui-Chen Chung, Taichung (TW); Shu-Mei Lin, Taichung (TW); Kuo-Chuan Chen, Taichung (TW); Tsai-Jen Hung, Taichung (TW)

(21) Appl. No.: 15/725,390

(22) Filed: Oct. 5, 2017

(30) Foreign Application Priority Data
Jul. 21, 2017 (TW) 106210815

(51) Int. Cl. B01D 61/14; B01D 71/02; A61K 38/39; A61K 35/28; B01D 11/02

(52) U.S. Cl. CPC B01D 61/145; B01D 71/024; A61K 38/39; B01D 2311/16; B01D 11/0292; B01D 2311/14; B01D 2311/103; A61K 35/28

(57) ABSTRACT

The present invention provides a device for separating and purifying the collagen Type 2 in chicken bones, comprising a liquid fluid container, a raw liquid container, a membrane separation tank, a mixing tube, two high pressure metering motors, a precooler, two preheaters, a temperature controller, two one-way valves, two inlet control valves and two outlet control valves. The liquid extract of defatted chicken bones discharged from the raw liquid container and the liquid CO2 discharged from the liquid fluid container can be mixed uniformly in the mixing tube, and then fed into the membrane separation tank. The membrane separation tank produces small-molecular-weight peptides and large-molecular-weight collagen Type 2 harmlessly and efficiently.

### BACKGROUND OF INVENTION

### 1. Field of the Invention

The present invention relates to a device for extracting chicken bone components, and more particularly to a device for separating and purifying the collagen Type 2 in chicken bones.

### 2. Description of Related Art

According to references, the Type 2 collagen structure in chicken bones contains chondroitin sulfate and glucosamine sulfate, the two substances can maintain the pH level of joints, and can mitigate ankylosis and the pain of arthritis. The chicken bones also contain chondroitin and hyaluronic acid, which can maintain the health of articular cartilage, and can enhance the human immune system. The collagen Type 2 can enter intestinal tract and maintain the integrity of intestinal tract, so as to enhance the effect of immune system, and to improve the health of digestive system

The known methods to extract collagen from chicken bones including using acid and basic solvents for hydrolytic reaction, this method uses a large amount of solvent, it is inapplicable to continuous mass industrialization; as well as using enzymatic hydrolysis, this method uses a lot of water as hydrolytic solvent, and the enzyme reaction shall be terminated by heat treatment, the protein is denatured in the process, and only small-molecular-weight peptide is obtained, such as gelatine substance.

Secondly, U.S. Pat. No. 6,838,440 B2, the chicken bones are dried at a low temperature, crushed and pulverized to obtain a low-concentration and low-purity dried chicken bone product, which has not been extracted, the taste acceptance is low, and a high dose shall be taken for appropriate effect. U.S. Pat. No. 4,804,745 also uses enzyme to hydrolyze protein to obtain peptide medicament for arthritis. U.S. Pat. No. 6,323,319 uses enzymatic hydrolysis and adjusts pH value to separate the collagen Type 2 from chicken bones by precipitation. The collagen is hydrolyzed basically by basic, acid or enzymatic hydrolysis, a large amount of solvent is used in the process, the economic cost is high. In addition, the other prior arts such as, the chicken bones and chicken feet are boiled in a large amount of high-pressure hot water, after heated hydrolytic reaction, the micromolecular peptide and glutin are dried by concentration, this technology cannot separate and purify high-purity collagen Type 2. Or, the chondroitin sulfate is extracted from chicken bones, and the chicken bone extract is obtained by heating, adsorbed and eluted by macroporous resin to obtain chondroitin sulfate, after the extraction in a large amount of hot water, the separated chondroitin sulfate is leached out by a large amount of salt solution, the process flow costs much time, and a lot of hot water and saline solution is used as solvent, concentration and drying are required after separation, the energy is consumed and the environmental protection effect is poor. Or, the collagen component in chicken bones is hydrolyzed by alkali protease and compound protease, deodorized by activated carbon and purified by membrane ultrafiltration. A large amount of water is used as solvent in the process, the work process is complicated.

In other words, the known methods to extract collagen from chicken bones have some defects, such as failing to obtain high-purity collagen Type 2, low taste acceptance, energy consumption, poor environmental protection effect, complex work process, or high consumption of solvent and high economic cost.

### SUMMARY OF THE INVENTION

The primary objective of the present invention is to provide a device for separating and purifying the collagen Type 2 in chicken bones, which can separate and purify high-purity collagen Type 2 and micromolecular peptides efficiently, the process is simple, and it does not consume energy, the environmental protection effect is perfect, and the economic value is high.

In order to attain the aforesaid purposes, the present invention provides a device for separating and purifying the collagen Type 2 in chicken bones, comprising a liquid fluid container for holding and supplying liquid CO₂; a raw liquid container for holding and supplying the liquid extract of defatted chicken bones; a membrane separation tank connected to the liquid fluid container and raw liquid container, it contains a filtering membrane for separating and purifying the small-molecular-weight peptides and large-molecular-weight collagen Type 2 from the liquid extract of defatted chicken bones; an electric heater for heating liquid CO₂ and the liquid extract of defatted chicken bones; a mixing tube connected to the liquid fluid container, raw liquid container and membrane separation tank, for mixing the liquid extract of defatted chicken bones and liquid CO₂ uniformly before they are fed in the membrane separation tank; two high pressure metering motors connected to the liquid fluid container, raw liquid container and mixing tube respectively, for feeding the liquid extract of defatted chicken bones and liquid CO₂ into the mixing tube; a precooler located between the liquid fluid container and high pressure metering motor; two preheaters connected to the two high pressure metering motors and mixing tube respectively; a temperature controller connected to the electric heater of the membrane separation tank; two one-way valves connected to the two preheaters and mixing tube respectively, so that the liquid extract of defatted chicken bones and liquid CO₂ only flow into the mixing tube; two inlet control valves located between the two one-way valves and mixing tube respectively, for controlling the entry of liquid extract of defatted chicken bones and liquid CO₂ into the mixing tube respectively; two outlet control valves connected to the membrane separation tank respectively, for controlling the membrane separation tank to discharge the retentate of macromolecular collagen Type 2 or the permeate of small-molecular-weight peptides respectively; controlling the opening of the inlet and outlet control valves, and controlling the volumetric flow rate ratio of permeate to retentate in a certain range.

### BRIEF DESCRIPTION OF THE DRAWINGS

FIG. 1 is the system diagram of a preferred embodiment of the present invention.

FIG. 2(A) is the HPLC analysis spectrum of liquid extract of defatted chicken bones in a preferred embodiment of the present invention.

FIG. 2(B) is the HPLC analysis spectrum of macromolecular collagen Type 2 retentate after separation and purification in a preferred embodiment of the present invention.

### DETAILED DESCRIPTION OF THE INVENTION

A preferred embodiment of the present invention is detailed in graphs as follows:

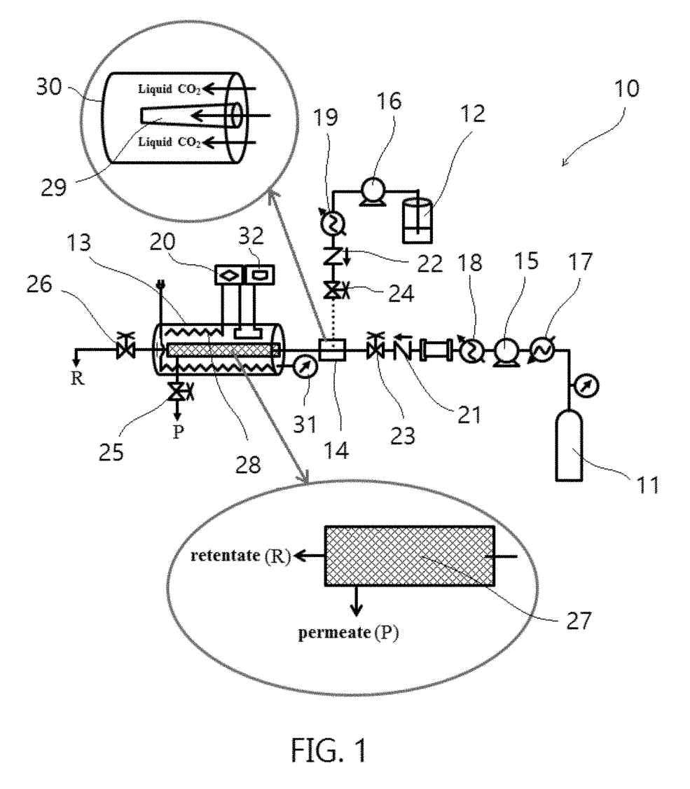

As shown in FIG. 1, the device for separating and purifying the collagen Type 2 in chicken bones 10 of a preferred embodiment of the present invention comprises a liquid fluid container 11, a raw liquid container 12, a membrane separation tank 13, a mixing tube 14, two high pressure metering motors 15, 16, a precooler 17, two preheaters 18, 19, a temperature controller 20, two one-way valves 21, 22, two inlet control valves 23, 24 and two outlet control valves 25, 26.

The liquid fluid container 11 stores and supplies liquid CO₂ fluid.

The raw liquid container 12 holds and supplies the liquid extract of defatted chicken bones. The liquid extract of defatted chicken bones is made by mixing the chicken bones with equivalent distilled water, the mixture is extracted, the residue is filtered, and the fat component is removed by refrigerated centrifugation.

The membrane separation tank 13 is a stainless steel tube in inside diameter of 0.036 m˜0.125 m and in height of 1.0 m, connected to the liquid fluid container 11 and raw liquid container 12. It contains a filtering membrane 27, which is Carbosep M2 or M8 molecular weight 15 kD or 50 kD cut-off ZrO₂/TiO₂ ceramic ultrafiltration membrane produced by France Novasep company, for separating and purifying small-molecular-weight peptides and large-molecular-weight collagen Type 2 from the liquid extract of defatted chicken bones, and an electric heater 28 for heating the liquid CO₂ and liquid extract of defatted chicken bones.

The mixing tube 14 is located among the liquid fluid container 11, raw liquid container 12 and membrane separation tank 13, comprising an inner tube 29 and an outer tube 30. The inner tube 29 is connected to the raw liquid container 12, and the outer tube 30 is connected to the liquid fluid container 11, for mixing the liquid extract of defatted chicken bones and liquid CO₂ uniformly before they are fed into the membrane separation tank 13.

The two high pressure metering motors 15, 16 are connected to the liquid fluid container 11, raw liquid container 12 and mixing tube 14 respectively, for feeding the liquid extract of defatted chicken bones and liquid CO₂ into the mixing tube 14.

The precooler 17 is located between the liquid fluid container 11 and high pressure metering motor 15.

The two preheaters 18, 19 are located between the two high pressure metering motors 15 and mixing tube 14 respectively.

The temperature controller 20 is connected to the electric heater 28 in the membrane separation tank 13 for controlling the heating temperature of the electric heater 28.

The two one-way valves 21, 22 are connected to the two preheaters 18, 19 and mixing tube 14 respectively, for making the liquid extract of defatted chicken bones and liquid CO₂ only flow into the mixing tube 14.

The two inlet control valves 23, 24 are located between the two one-way valves 21, 22 and mixing tube 14 respectively, for controlling the liquid extract of defatted chicken bones and liquid CO₂ to or not to enter the mixing tube 14 respectively.

The two outlet control valves 25, 26 are connected to the membrane separation tank 13 respectively, for controlling the membrane separation tank 13 to discharge the retentate of macromolecular collagen Type 2 or the permeate of small-molecular-weight peptides.

In addition, the device comprises a pressure transducer 31 located in one end of the membrane separation tank 13, for regulating the pressure in the membrane separation tank 13. A digital temperature indicator 32 electrically connected to the temperature controller 18, for displaying the temperature in the membrane separation tank 13. The opening of the inlet and outlet control valves 23, 24, 25, 26 are controlled, and the volumetric flow rate ratio of permeate to retentate can be controlled in a certain range.

Thereby, the operation mode, characteristics and effect of the device for separating and purifying the collagen Type 2 in chicken bones 10 of the present invention are described below:

First, the liquid fluid container 11 is opened, working with the high pressure metering motor 15, so that the liquid CO₂ enters the mixing tube 14 through the one-way valve 21 at volumetric flow rate of 2-4 L/hr. The liquid extract of defatted chicken bones in the raw liquid container 12 is fed into the mixing tube 14 through the one-way valve 22 at volumetric flow rate of 200-500 mL/hr by the high pressure metering motor 16, so that the liquid CO₂ and liquid extract of defatted chicken bones are mixed uniformly in the mixing tube 14. The two outlet control valves 25, 26 are turned on simultaneously to control the pressure in the membrane separation tank 13 at 150-200 psi, and the temperature in the membrane separation tank 13 is controlled at 40-60° C. by temperature controller 20, and the inlet and outlet control valves 23, 24, 25, 26 are controlled, so as to keep the volumetric flow rate ratio of permeate (P, small-molecular-weight peptides) to retentate (R, retentate of macromolecular collagen Type 2) discharged by the two outlet control valves 25, 26 at 4.0-6.0/1.

After the separation and purification of the membrane separation tank 13, the outlet control valve 25 is turned on to collect the separated permeate (P), which is generally composed of small-molecular-weight peptides, such as chondroitin, glycosaminoglycans (GAGS), glucosamine, hyaluronic acid and amino acids. The outlet control valve 26 is turned on to collect retentate (R), which is generally composed of large-molecular-weight substance collagen Type 2 which cannot pass through the filtering membrane 27.

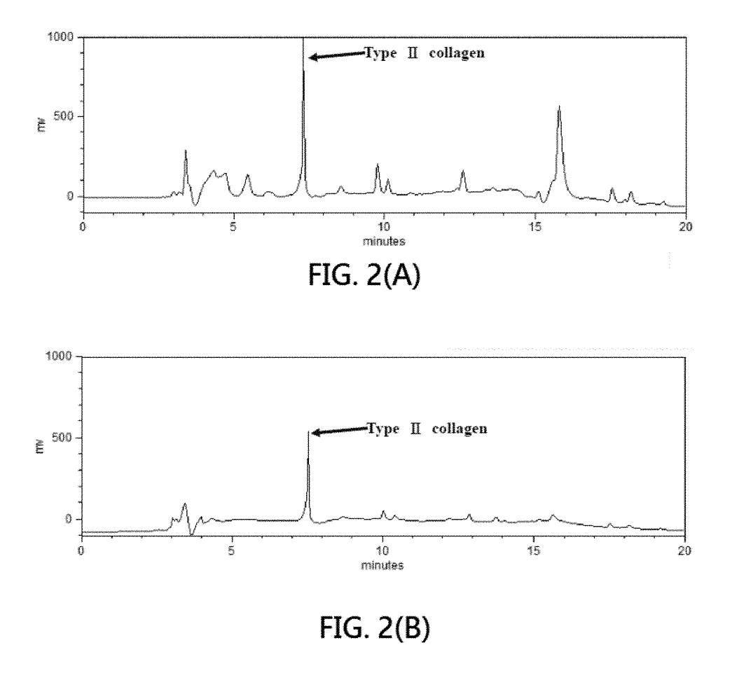

The BioSep-SEC-S2000 (7.8 mm×300 mm, 5 μm) chromatographic column and HPLC quantitative analysis instrument are used, the mobile phase is 0.15 mol/L KH₂PO₄ buffer solution (pH=4.7), the flow velocity is 1.0 ml/min, the UV detection wavelength is 210 nm, the tubular column temperature is room temperature, the concentration of collagen Type 2 in the sample is quantified, and the liquid extract of defatted chicken bones is analyzed, as shown in FIG. 2(A). The retentate (R) sample separated and purified by the membrane separation tank 13 is analyzed, as shown in FIG. 2(B):

The chicken bones are dried at a low temperature and pulverized, mixed with water or other solvents for extraction, the product contains all constituents of chicken bones, such as macromolecular collagen Type 2 and micromolecular peptides, e.g. chondroitin, glucosamine, hyaluronic acid and amino acid. As shown in FIG. 2(A), the liquid extract sample of defatted chicken bones is analyzed by HPLC, the collagen Type 2 and micromolecular peptides are obtained simultaneously. When the raw chicken bones are hydrolyzed by acid and basic solvents or enzyme, the micromolecular peptides are obtained, such as chondroitin, hyaluronic acid and amino acid. This process cannot obtain collagen Type 2. When the raw chicken bones are dried at a low temperature, the pulverized powder product only contains low concentration collagen Type 2; when the collagen Type 2 is precipitated by separation by chemical precipitation agent, e.g. trichloroacetic acid, the impurities are removed by dialysis in a large amount of solvent (e.g. DI water, buffer solution) with semipermeable dialysis membrane (e.g. Spectra/Por Dialysis Membrane), the purified collagen Type 2 is left. This method cannot obtain micromolecular peptides, such as chondroitin, glucosamine, hyaluronic acid and amino acid.

As shown in FIG. 2(B), the high concentration and purity macromolecular collagen Type 2 from the complete liquid extract of defatted chicken bones is separated and purified in the retentate (R) of the membrane separation tank 13, and the high concentration and purity micromolecular chondroitin, glycosaminoglycans, glucosamine, hyaluronic acid and amino acid can be separated and purified in the permeate (P) of the membrane separation tank 13.

Therefore, the device for separating and purifying the collagen Type 2 in chicken bones of the present invention uses environmentally friendly liquid CO₂ fluid and physical method of membrane separation (membrane separation tank), high concentration macromolecular collagen Type 2 and micromolecular chondroitin, glycosaminoglycans, glucosamine, hyaluronic acid and amino acid can be obtained simultaneously and efficiently, the process is simple, and it does not consume energy, the environmental protection effect is good, and the economic value is high.

Although the invention has been explained in relation to its preferred embodiment, it is to be understood that many other possible modifications and variations can be made without departing from the spirit and scope of the invention as hereinafter claimed.

What is claimed is:

1. A device for separating and purifying the collagen Type 2 in chicken bones, comprising :
a liquid fluid container for holding and supplying liquid CO₂;
a raw liquid container for holding and supplying the liquid extract of defatted chicken bones, the liquid extract of defatted chicken bones in the raw liquid container is obtained by mixing the chicken bones with equivalent distilled water, the mixture is extracted, the residue is filtered, and the fat constituent is removed by refrigerated centrifugation;
a membrane separation tank connected to the liquid fluid container and raw liquid container, it contains a filtering membrane for separating and purifying small-molecular-weight peptides and large-molecular-weight collagen Type 2 from the liquid extract of defatted chicken bones, and an electric heater for heating liquid CO₂ and liquid extract of defatted chicken bones;
a mixing tube connected to the liquid fluid container, raw liquid container and membrane separation tank, for mixing the liquid extract of defatted chicken bones and liquid CO₂ uniformly before they are fed into the membrane separation tank, the mixing tube comprises an inner tube and an outer tube, the inner tube is connected to raw liquid container, and the outer tube is connected to the liquid fluid container, and the volumetric flow rate of liquid CO₂ fed in the mixing tube is 2-4 L/hr, the volumetric flow rate of liquid extract of defatted chicken bones fed in the mixing tube is 200-500 mL/hr, the liquid CO₂ and liquid extract of defatted chicken bones are mixed in the mixing tube till saturated state;
two high pressure metering motors connected to the liquid fluid container, raw liquid container and mixing tube respectively, for feeding the liquid extract of defatted chicken bones and liquid CO₂ into the mixing tube;
a precooler located between the liquid fluid container and high pressure metering motor;
two preheaters connected to the two high pressure metering motors and mixing tube respectively;
a temperature controller connected to the electric heater of the membrane separation tank;
two one-way valves connected to the two preheaters and mixing tube respectively, making the liquid extract of defatted chicken bones and liquid CO₂ only flow into the mixing tube;
two inlet control valves located between the two one-way valves and mixing tube respectively, for controlling the liquid extract of defatted chicken bones and liquid CO₂ to or not to enter the mixing tube respectively;
two outlet control valves connected to the membrane separation tank respectively, for controlling the membrane separation tank to discharge retentate (R) of macromolecular collagen Type 2 or permeate (P) of small-molecular-weight peptides;
a pressure transducer located in one end of the membrane separation tank for regulating the pressure in the membrane separation tank; and
the opening of the inlet and outlet control valves is controlled, and the volumetric flow rate ratio of permeate to retentate can be controlled in a certain range.

2. The device defined in claim 1, wherein the membrane separation tank is a stainless steel tube, the filtering membrane is a ceramic ultrafiltration membrane.

3. The device defined in claim 2, wherein the filtering membrane can be molecular weight 15 kD or 50 kD cut-off ZrO₂/TiO₂.

4. The device defined in claim 1, wherein the pressure transducer is regulated to keep the pressure in the membrane separation tank at 150-200 psi, and the temperature in the membrane separation tank is controlled by the temperature controller at 40-60° C.

5. The device defined in claim 4, wherein the opening of the inlet and outlet control valves is controlled, the volumetric flow rate ratio of permeate to retentate is kept at 4.0-6.0/1.
